(19) United States
(12) Patent Application Publication
(10) Pub. No.: US 2018/0147155 A1
(43) Pub. Date: May 31, 2018

(54) TRANSDERMAL DEVICE AND TRANSDERMAL PATCH

(71) Applicants: Atsumi YAMABE, Chiba (JP); Toshihiro KANEMATSU, Kanagawa (JP)

(72) Inventors: Atsumi YAMABE, Chiba (JP); Toshihiro KANEMATSU, Kanagawa (JP)

(21) Appl. No.: 15/815,012

(22) Filed: Nov. 16, 2017

(30) Foreign Application Priority Data
Nov. 28, 2016 (JP) 2016-230302

(51) Int. Cl. A61K 9/70; A61N 1/04

(52) U.S. Cl. CPC A61K 9/7084; A61N 1/327; A61N 1/0428; A61K 9/7061

(57) ABSTRACT

A transdermal device is provided. The transdermal device includes a transdermal patch, a sheet-shaped electrode stacked overlying one surface of the transdermal patch, and a power source connected to the sheet-shaped electrode. The transdermal patch includes a carrier layer. The carrier layer comprises a hollow structure and an external composition. The hollow structure comprises a plurality of cells partitioned by an insulator and has hollow portions penetrating through one plane surface to the other plane surface of the hollow structure. The external composition comprises a dispersion liquid comprising a nanoparticle containing an active ingredient.

### CROSS-REFERENCE TO RELATED APPLICATIONS

This patent application is based on and claims priority pursuant to 35 U.S.C. § 119(a) to Japanese Patent Application No. 2016-230302, filed on Nov. 28, 2016 in the Japan Patent Office, the entire disclosure of which is hereby incorporated by reference herein.

### BACKGROUND

### Technical Field

The present disclosure relates to a transdermal device and a transdermal patch.

### Description of the Related Art

As means for transdermally or transmucosally introducing drugs to living bodies, transdermal devices using iontophoresis are known.

In transdermal or transmucosal drug administration, drug transport efficiency and drug permeability to the skin are required to be high. To meet this requirement, the transdermal devices may increase drug dosage and dosing rate by increasing the level of current or voltage. However, this results in an increase in stimulation on the skin, possibly causing burns.

In most conventional transdermal patches, the member that holds or contains a drug is made of a material (e.g., unwoven fabric, porous material, sponge, gel-like substance) which exhibits no anisotropy in the direction of electric field. Therefore, the supplied current is consumed by the member itself while degrading current efficiency. As a result, drug transport efficiency (i.e., electrophoretic property) and drug permeability to the skin may be insufficient.

The shape and material of the member that holds or contains a drug affect not only the supplied current efficiency and electrophoretic property but also followability to expansion and contraction of the skin. Because the patch is directly applied to the skin, uncomfortable feeling may occur unless the patch can follow the expansion and contraction of the skin caused due to the movement of the body and also the patch excels in adhesiveness. If the followability is poor, the patch may come off when applied to the skin near joints such as elbows and knees.

Some of the conventional members cannot follow the expansion and contraction of the skin caused by the movement of the body. If the followability is poor, the air may enter from the applied surface to cause insulation, so that satisfactory electrophoretic property and drug permeability to the skin may not be obtained.

### SUMMARY

In accordance with some embodiments of the present invention, a transdermal device is provided. The transdermal device includes a transdermal patch, a sheet-shaped electrode stacked overlying one surface of the transdermal patch, and a power source connected to the sheet-shaped electrode. The transdermal patch includes a carrier layer. The carrier layer comprises a hollow structure and an external composition. The hollow structure comprises a plurality of cells partitioned by an insulator and has hollow portions penetrating through one plane surface to the other plane surface of the hollow structure. The external composition comprises a dispersion liquid comprising a nanoparticle containing an active ingredient.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a schematic view of a transdermal device in accordance with some embodiments of the present invention;

FIG. 1B is a cross-sectional view of the transdermal device illustrated in FIG. 1A;

FIG. 2 is a schematic view of a transdermal device in accordance with some embodiments of the present invention;

FIG. 3 is a schematic view of a transdermal device in accordance with some embodiments of the present invention; and

FIGS. 4A and 4B are schematic views of a hollow structure included in a transdermal device in accordance with some embodiments of the present invention.

The accompanying drawings are intended to depict example embodiments of the present invention and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

Embodiments of the present invention are described in detail below with reference to accompanying drawings. In describing embodiments illustrated in the drawings, specific terminology is employed for the sake of clarity. However, the disclosure of this patent specification is not intended to be limited to the specific terminology so selected, and it is to be understood that each specific element includes all technical equivalents that have a similar function, operate in a similar manner, and achieve a similar result.

For the sake of simplicity, the same reference number will be given to identical constituent elements such as parts and materials having the same functions and redundant descriptions thereof omitted unless otherwise stated.

Within the context of the present disclosure, if a first layer is stated to be “overlaid” on, or “overlying” a second layer, the first layer may be in direct contact with a portion or all of the second layer, or there may be one or more intervening layers between the first and second layer, with the second layer being closer to the substrate than the first layer.

In accordance with some embodiments of the present invention, a transdermal device is provided that has high followability to the skin and mucous membrane, high electrophoretic property of drugs, and high drug permeability to the skin.

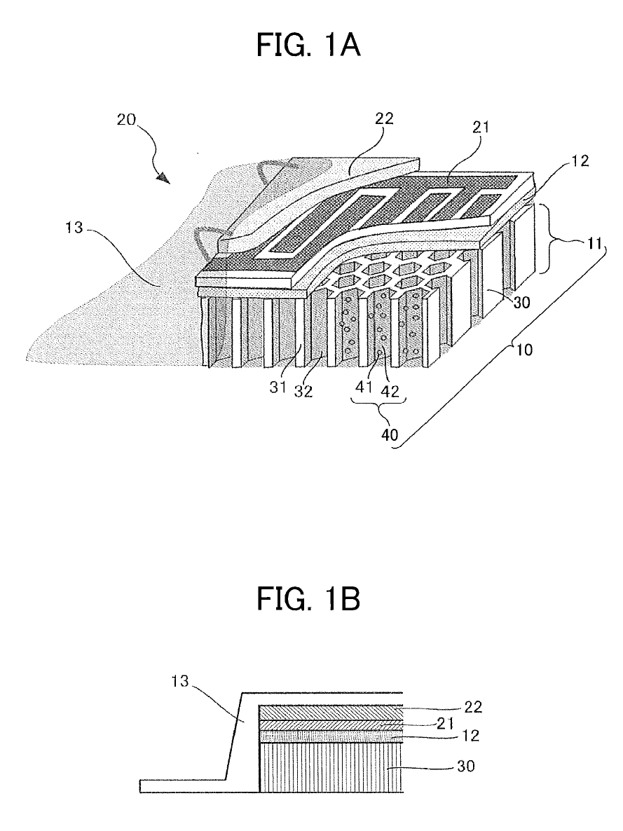

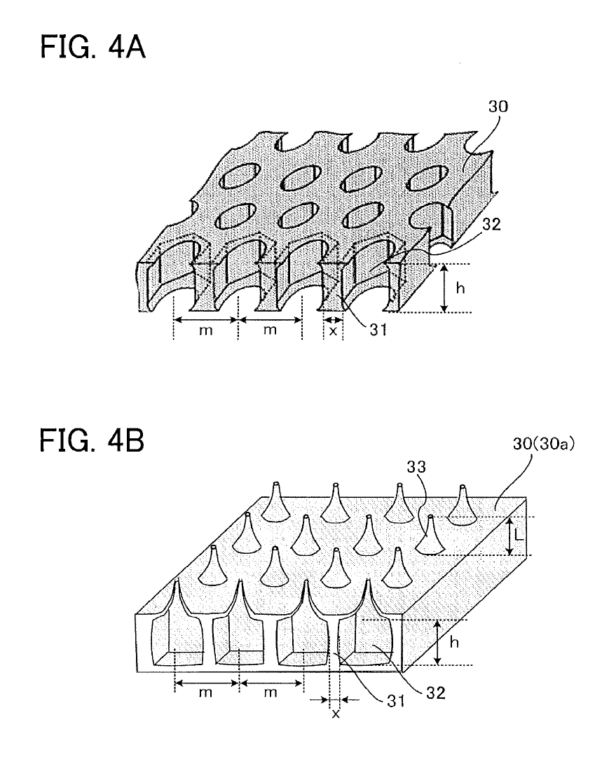

FIGS. 1A, 1B, 2, and 3 are schematic views illustrating transdermal devices in accordance with some embodiments of the present invention. FIGS. 4A and 4B are schematic views each illustrating a hollow structure of a carrier layer in a transdermal patch or device in accordance with some embodiments of the present invention.

As illustrated in FIGS. 1A, 1B, 2, and 3, a transdermal device 20 includes a transdermal patch 10, a sheet-shaped electrode 21, and a power source 22. The transdermal patch 10 comprises a carrier layer 11 carrying an external composition 40. The electrode 21 is stacked overlying one surface of the transdermal patch 10. The power source 22 is connected to the electrode 21.

The external composition 40 comprises a dispersion liquid comprising a nanoparticle 41 and a dispersion medium 42. The nanoparticle 41 is containing an active ingredient.

The carrier layer 11 may be brought into direct contact with the skin or mucous membrane (hereinafter simply and collectively referred to as “the skin”). Alternatively, the carrier layer 11 may be brought into indirect contact with the skin via a conductive member interposed between the carrier layer 11 and the skin. When the external composition 40 has a low viscosity, a liquid-permeable member (e.g., microporous film) may be interposed therebetween.

The sheet-shaped electrode 21 may be stacked on the carrier layer 11 via an adhesive layer 12.

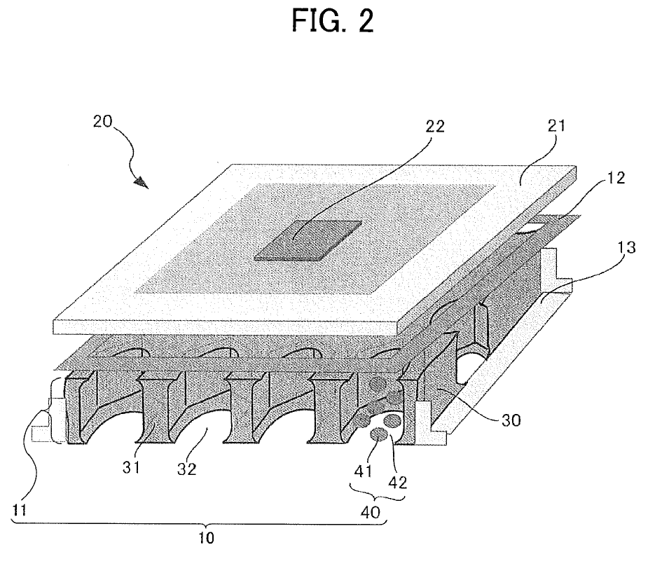

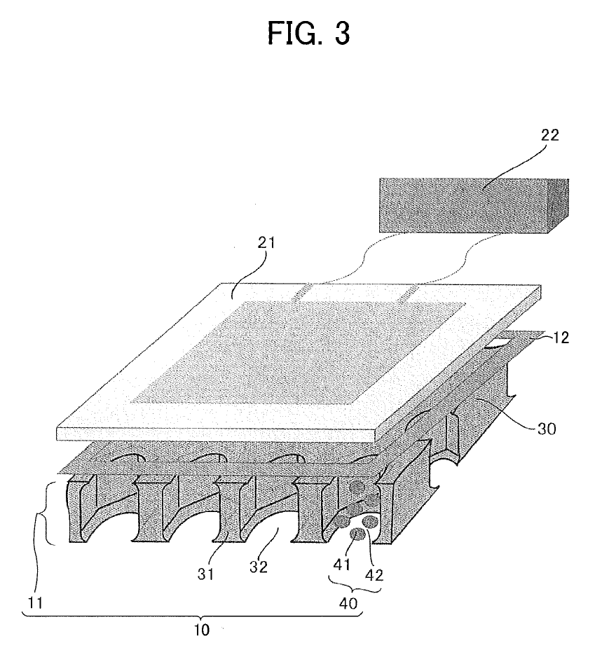

The power source 22 may be stacked on the electrode 21, as illustrated in FIGS. 1A, 1B, and 2. Alternatively, the power source 22 may be connected to an extraction electrode of the electrode 21 via an alligator clip or the like, as illustrated in FIG. 3.

The electrode 21 and the power source 22 give charge to the carrier layer 11, and ingredients of the external composition 40 carried by the carrier layer 11 are transferred to the skin.

### [1] Carrier Layer (Hollow Structure)

The carrier layer 11 comprises a hollow structure 30 comprising multiple cells 32 partitioned by an insulator.

The hollow structure 30 has hollow portions penetrating through one plane surface to the other plane surface of the hollow structure 30. Therefore, the hollow structure 30 exhibits good followability in the planar direction. Since there exists no partition wall extending in the planar direction, excellent flexibility is exhibited in the planar direction, although there exist partition walls extending in a direction connecting both plane surfaces of the hollow structure 30.

Thus, the hollow structure 30 exhibits high followability to expansion and contraction of the skin.

The cells 32 in the hollow structure 30 of the carrier layer 11 provide flow paths for the external composition 40. Preferably, the multiples cells 32 are arranged side by side in a regular manner. By such an arrangement, current efficiency is improved so that ingredients of the external composition 40 can easily reach the skin.

In the hollow structure 30, each of the cells 32 forms a flow path having a substantially linear shape. As a result, the cells 32 exhibit anisotropy in the electric field so that the active ingredient contained in the nanoparticle 41 of the external composition 40 can effectively perform electrophoresis. Thus, transdermal absorptivity of the active ingredient can be improved.

Preferably, the cells 32 have a height of from 10 to 2,000 μm. Here, the height refers to the length of the hollow structure 30 in a thickness direction, represented by “h” in FIGS. 4A and 4B.

In addition, preferably, the cells 32 have a pitch of from 5 to 500 μm. Here, the pitch refers to the distance between the centers of the adjacent cells 32, represented by “m” in FIGS. 4A and 4B.

The height and pitch can be appropriately adjusted according to the type of the external composition 40 to be carried by selecting appropriate manufacturing conditions and/or materials for the cells 32.

Preferably, a partition wall 31 that partitions the adjacent cells 32 have a thickness of from 0.1 to 30 μm. The thickness is represented by “x” in FIGS. 4A and 4B.

When the thickness of the partition wall 31 is less than 0.1 μm, it is difficult for the hollow structure 30 to carry the external composition 40 and the partition wall 31 gets easily broken. When the thickness of the partition wall 31 is in excess of 30 μm, it is difficult to deform the partition wall 31 and thus followability to the skin deteriorates.

The cells 32 can carry multiple types of external compositions 40 respectively containing different types of active ingredients.

In this case, the external compositions 40 may be separately carried by different cells 32 so as not to mix the different types of active ingredients and reduce their activity by mixing.

When there is no problem of such activity reduction caused due to mixing of active ingredients, the partition wall 31 can be replaced with a porous material so long as the current efficiency is not adversely affected. In this case, followability to the skin can be more improved.

As illustrated in FIGS. 1A, 2, and 3, the hollow structure 30 may be a honeycomb structure, but is not limited thereto.

The cross-sectional shape of the cells 32 may be either a circular shape as illustrated in FIG. 4A or a polygonal shape (including a hexagonal shape in the honeycomb structure).

The hollow structure 30 may comprise a micro-needle array 30a having hollow needles 33 on a surface contactable with the skin, as illustrated in FIG. 4B. The hollow needles 33 are communicated with the respective cells 32. FIG. 4B illustrates the micro-needle array 30a on the way of being manufactured (just has been released from a template, to be described in detail later). The micro-needle array 30a will be finished after openings have been formed by a mechanical process (cutting) so that the cells 32 are penetrating from one plane surface to the other plane surface thereof. The finished micro-needle array 30a can be applied to the transdermal device.

The micro-needle array 30a has multiple hollow needles 33. Preferably, the hollow needles 33 each have a length of from 1 to 200 μm. The length of each hollow needle 33 refers to the length between the base and distal end thereof, represented by “L” in FIG. 4B. When the length of the hollow needle 33 is from 1 to 200 μm, the hollow needle 33 can deliver the external composition 40 to the stratum corneum (horny layer), without reaching the dermal layer, from the hole disposed on the distal end thereof.

The inner diameter of the hole on the distal end of the hollow needle 33 can be appropriately adjusted within a range that the external composition 40 can be delivered to the stratum corneum (horny layer). For example, the inner diameter of the hole may be in the range of from 2 to 20 μm.

When the micro-needle array 30a is applied to the skin, in some cases, the hollow needles 33 cause buckling due to low strength thereof. To avoid such a problem, the hollow needles 33 may have a coating layer comprising biocompatible polymer on the outer and/or inner walls of the distal end region thereof.

### Method for Manufacturing Hollow Structure

The hollow structure may be manufactured by, for example, a method disclosed in JP-4678731-B (corresponding to JP-2007-098930-A) or JP-4869269-B (corresponding to JP-2009-214374-A).

JP-4678731-B discloses a method for manufacturing a honeycomb structure in the following manner.

(1) On a member (hereinafter “template”) having independent recesses, a base material is disposed so as to cover the recesses. The base material comprises a protective material and a material (e.g., uncured ultraviolet-curable resin) applied to the protective material.

(2) The environment surrounding the base material and the template are decompressed (vacuumed) so that the gas inside the recesses relatively generate a pressure and, at the same time, the pressure expands the base material disposed on the recesses, thereby forming a hollow structure (e.g., honeycomb structure, micro-needle array).

(3) At the time the partition wall of the cells has grown to have a desired height, an energy ray (e.g., ultraviolet ray) is emitted thereto to cure the base material.

(4) The resulting hollow structure is detached from the template.

(5) Since the detached hollow structure has a shape such that one plane surface is closed, openings are formed by a mechanical process (i.e., cutting) so that the cells are penetrating though one plane surface to the other plane surface.

The height of the cells and the thickness of the partition wall of the cells can be controlled by controlling pressure during the manufacturing process and adjusting mechanical properties (e.g., viscosity, strength, and breaking elongation) of the base material.

The template has independent recesses. The shape of the cells 32 is determined depending on the arrangement of the recesses. For example, when the recesses are in a zigzag arrangement, the shape of the cells becomes hexagonal. When the recesses are in a lattice arrangement, the shape of the cells becomes quadrangular. The pitch (distance) between the centers of the adjacent recesses is equivalent to the pitch between the centers of the adjacent cells.

Examples of the material used for the template include, but are not limited to, nickel, silicon, stainless steel, and copper.

To tightly attach the base material to the template, an attachment jig can be used. Examples of the attachment jig include, but are not limited to, a roller member.

It is preferable that a pressure control is performed so that the base material will not excessively enter the openings on the template. In addition, it is preferable that the base material is attached to the template from the edge part thereof so that bubbles do not enter other parts.

When the hollow structure is detached from the template, a detachment jig can be used. For example, the hollow structure may be held with a tweezers-like jig and drawn up to be detached from the template.

The base material comprises the protective material to which a material is applied. The protective material protects the gas from leaking during the decompression process and relaxes stress concentration during the detachment process so as to prevent defective detachment. In a case in which the material applied to the protective material is a ultraviolet-curable resin, the protective material preferably comprises an ultraviolet-permeable material, such as flexible plastics (e.g., PET (polyethylene terephthalate), PE (polyethylene)).

### Material of Hollow Structure

The material of the hollow structure may be an insulating material having low irritation and toxicity to living bodies. Examples of such a material include biocompatible materials, thermoplastic resins, polymer materials, ultraviolet curable resins, and polydimethylsiloxane.

Specific examples of the biocompatible materials include, but are not limited to: biological-origin soluble substances such as chitosan, collagen, gelatin, hyaluronic acid (HA), alginic acid, pectin, carrageenan, chondroitin (sulfate), dextran (sulfate), polylysine, carboxymethyl chitin, fibrin, agarose, pullulan, and cellulose; biocompatible substances such as polyvinylpyrrolidone (PVP), polyethylene glycol (PEG), polyvinyl alcohol (PVA), hydroxypropyl cellulose (HPC), hydroxyethyl cellulose (HEC), hydroxypropyl methylcellulose (HPMC), carboxymethyl cellulose sodium, polyalcohol, gum arabic, alginate, cyclodextrin, dextrin, glucose, fructose, starch, trehalose, glucose, maltose, lactose, lactulose, fructose, turanose, melitose, melezitose, dextran, sorbitol, xylitol, palatinit, polylactic acid, polyglycolic acid, polyethylene oxide, polyacrylic acid, polyacrylamide, polymethacrylic acid, and polymaleic acid; derivatives of the above substances; and mixtures of the above substances.

Specific examples of the thermoplastic resins include, but are not limited to: polyolefin such as polyethylene, polypropylene, and ethylene-α-olefin copolymer; polyamide; polyurethane; polyester such as polyethylene terephthalate, polybutylene terephthalate, polycyclohexane terephthalate, and polyethylene-2,6-naphthalate; and fluororesin such as PTFE (polytetrafluoroethylene) and ETFE (ethyl enetetrafluoroethyl ene).

In a case in which the material is difficult to be formed into the hollow structure, a surfactant can be introduced to the material.

Specific examples of the surfactant include, but are not limited to: anionic surfactants such as calcium stearate, magnesium stearate, and sodium lauryl sulfate; cationic surfactants such as benzalkonium chloride, benzethonium chloride, and cetylpyridinium chloride; and non-ionic surfactants such as glyceryl monostearate, sucrose fatty acid esters, polyoxyethylene hardened castor oil, and polyoxyethylene sorbitan fatty acid esters.

In a case in which the material is a water-soluble material (e.g., gelatin), an insolubilizing agent may be added thereto to improve water resistance.

Specific examples of the insolubilizing agent include, but are not limited to: organic compounds such as quinones and ketones; and inorganic compounds such as ferric compounds and chrome. In particular, these organic compounds having a pH around 8 and these inorganic compounds having a pH around 4.5 are preferable. Organic compounds are more preferable because they do not cause metallic allergy even when applied to the skin.

Alternatively, the water-soluble material can be insolubilized by being exposed to heat, γ-ray, etc., without introducing any insolubilizing agent.

To prevent the hollow structure from being destroyed when detached from the template, the following adhesive-force-variable materials types (1) to (3) may be used.

(1) Materials being solid when the material of the hollow structure is applied (extended) and liquid when the hollow structure is detached.

Examples of such materials include, but are not limited to, a hot-melt adhesive that transits from solid to liquid upon heating. The hot-melt adhesive remaining in the hollow structure 30 can be used for bonding to other members.

(2) Material being solid when the material of the hollow structure is applied (extended) and gaseous or liquid when the hollow structure is detached.

Examples of such materials include, but are not limited to, water (e.g., vapor, ice). Water may be applied to a substrate and cooled by a temperature controller to become ice before the material of the hollow structure is applied thereto, so that the adhesive force of water to the material of the hollow structure is improved. After the hollow structure 30 has been formed, the ice may be removed by being liquefied by being heated by a temperature controller, followed by heat-drying. The ice may be removed by further being heated to become vapor.

(3) Material being viscous when the material of the hollow structure is applied (extended) and non-viscous when the hollow structure is detached.

Examples of such materials include, but are not limited to, materials that change viscoelasticity before and after exposure to ultraviolet rays, such as an adhesive material used for a dicing tape that prevents chips from breaking up at the time of dicing of a silicon wafer. Such an adhesive material can be cured by exposure to ultraviolet ray and detached when the adhesion strength has been lowered.

### [2] External Composition

The external composition comprises a dispersion liquid comprising a nanoparticle containing an active ingredient.

In the present disclosure, the external composition refers to a composition containing drugs, quasi-drugs, or cosmetics, directly applicable to the skin or mucous membrane.

Examples of the external composition include, but are not limited to, medicated cosmetics, nutritional supplements, diagnostic drugs, and therapeutic drugs.

The dispersion medium of the dispersion liquid may be water, an electrolyte aqueous solution, or an organic solvent. Among these, water and an electrolyte aqueous solution are preferable, and an electrolyte aqueous solution is more preferable.

The electrolyte is not particularly limited so long as it is a biocompatible material. Examples of the biocompatible material include, but are not limited to, sodium chloride, potassium chloride, sodium bromide, potassium bromide, calcium chloride, and calcium bromide.

The type of the dispersion medium can be appropriately selected in accordance with the type of the nanoparticle, the current value, and/or the parts (e.g., stratum corneum, dermal layer, blood) to which the nanoparticle is to be delivered.

### Nanoparticle

Preferably, the nanoparticle has a particle diameter of 500 nm or less, more preferably from 10 to 100 nm, and most preferably from 40 to 80 nm.

When the particle diameter is less than 10 nm, although permeability to the skin is high, the desired effect may not be obtained by diffusion of the nanoparticle. When the particle diameter is in excess of 100 nm, permeability to the skin may deteriorate because the nanoparticle is larger than the pores of the skin. It is known that, in iontophoresis, an active ingredient can permeate the skin by opening tight junctions by giving a current. However, the desired permeability cannot be achieved when the particle diameter of the active ingredient is too large.

The nanoparticle is not particularly limited so long as it is capable of containing an active ingredient and performing electrophoresis. Examples of the nanoparticle include liposome, micelle, and organic nanotube.

The nanoparticle may be made of a biocompatible material capable of forming nanosized liposome or micelle. Specific examples of such a material include, but are not limited to, poly(p-dioxanone) (“PPDX”), poly(lactide-co-glycolide) (“PLGA”), polycaprolactone, polylactic acid, polyanhydride, polyorthoester, polyether ester, polyesteramide, polyamide, polyethylene glycol, and polybutyric acid.

The concentration of the nanoparticle is determined depending on the type of the active ingredient contained in the nanoparticle. For example, the concentration of the contained active ingredient may be adjusted to within the range of from 0.1 to 100 mM. It is difficult to produce the dispersion liquid having a concentration exceeding 100 mM.

By adjusting the concentration of the nanoparticle to within an appropriate range, the desired effect can be exerted. The concentration of the contained active ingredient is preferably in the range of from 0.5 to 15 mM, more preferably from 1 to 10 mM, and most preferably from 1 to 7 mM.

### Active Ingredient

The active ingredient contained in the nanoparticle can be appropriately selected according to the purpose.

For example, the active ingredient may comprise medicinal ingredients (particularly, polymer medicinal ingredients) as raw materials of medicated cosmetics. Specific examples of the medicinal ingredients include, but are not limited to: whitening ingredients such as ascorbic acid, vitamin C ethyl, vitamin C glycoside, ascorbyl palmitate, kojic acid, Rucinol, tranexamic acid, oil-soluble licorice extract, vitamin A derivatives, and placenta extract; anti-wrinkle ingredients such as retinol, retinoic acid, retinol acetate, retinol palmitate, EGF, cell culture extract, and acetyl glucosamine; blood circulation promoting ingredients such as tocopherol acetate, capsaicin, and nonanoic acid vanillylamide; dieting ingredients such as raspberry ketone, evening primrose extract, and seaweed extract; antimicrobial ingredients such as isopropylmethylphenol, Kankoh-so (i.e., cosmetic ingredients developed in Japan), and zinc oxide; vitamins such as vitamin D2, vitamin D3, and vitamin K; and sugars such as glucose, trehalose, and maltose. Specific examples of the polymer medicinal ingredients include, but are not limited to, physiologically active peptides and derivatives thereof, nucleic acid, oligonucleotide, and fragments of various antigenic proteins, bacterias, and viruses.

In addition, the following substances may be added for the purpose of accelerating transdermal absorption: non-ionic surfactants (e.g., glyceryl monostearate, sucrose fatty acid ester), water-soluble polymer compounds (e.g., carboxylic acid), water-soluble chelate agents (e.g., EDTA), aromatic carboxylic acid compounds (e.g., salicylic acid and derivatives thereof), aliphatic carboxylic acid compounds (e.g., capric acid, oleic acid), bile acid salts, propylene glycol, hydrogenated lanoline, isopropyl myristate, diethyl sebacate, urea, lactic acid, and Azone.

Furthermore, an ultraviolet absorbing agent or an ultraviolet scattering agent may be contained in the nanoparticle.

Specific examples of the ultraviolet absorbing agent include, but are not limited to: cinnamic-acid-based ultraviolet absorbers such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxycinnamate, isopropyl-p-methoxycinnamate, isoamyl-p-methoxycinnamate, octyl-p-methoxycinnamate, 2-ethoxyethyl-p-methoxycinnamate, cyclohexyl-p-methoxycinnamate, ethyl-α-cyano-β-phenyl cinnamate, 2-ethylhexyl-α-cyano-β-phenyl cinnamate, and glyceryl mono-2-ethylhexanoyl-di(p-methoxycinnamate); benzophenone-based ultraviolet absorbers such as 2,4-dihydroxybenzophenone, 2,2′-dihydroxy-methoxybenzophenone, 2,2′-dihydroxy-4,4′-dimethoxybenzophenone, 2,2′,4,4′-tetrahydroxydibenzophenone, 2-hydroxy-4-methoxybenzophenone, 2-hydroxy-4-methoxy-4′-methyl benzophenone, 2-hydroxy-4-methoxybenzophenone-5-sulfonate, 4-phenyl benzophenone, 2-ethylhexyl-4′-phenyl-benzophenone-2-carboxylate, 2-hydroxy-4-n-octoxybenzophenone, and 4-hydroxy-3-carboxybenzophenone; p-aminobenzoic-acid-based ultraviolet absorbers such as PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester; salicylic-acid-based ultraviolet absorbers such as amyl salicylate, menthyl salicylate, homomethyl salicylate, octyl salicylate, phenyl salicylate, benzyl salicylate, and p-isopropanolphenyl salicylate; and 3-(4′-methyl benzylidene)-d-camphor, 3-benzylidene-d,l-camphor, urocanic acid, urocanic acid ethyl ester, octyl triazone, and 4-methoxy-4′-t-butyldibenzoylmethane.

Specific examples of the ultraviolet scattering agent include, but are not limited to, particulate titanium oxide, zinc oxide, and cerium oxide.

In addition, typical additives generally added to external compositions may also be contained in the nanoparticle. The type and addition amount can be appropriately selected within a range that the stability of the composition and the desired effect of the active ingredient are not adversely affected.

Specific examples of such additives include, but are not limited to, non-ionic polymers (e.g., guar gum, tamarind gum), cationic polymers (e.g., cationized cellulose, diallyldimethylammonium chloride polymer), anionic polymers (e.g., xanthane gum, sodium alginate), natural water-soluble compounds and derivatives thereof, surfactants, oily components, colorants, preservatives, chelate agents, antioxidants, moisturizing agents, lower alcohols, polyols, fragrances, refrigerants, and pH adjusters.

The surfactant is added for the purpose of emulsification, solubilization, and dispersion. Specific examples of the surfactant include, but are not limited to: non-ionic surfactants such as POE fatty acid ester, polyglycerin fatty acid ester, POE higher alcohol ether, and POE-POP block polymer; anionic surfactants such as fatty acid potassium, fatty acid sodium, higher alkyl sulfate salt, alkyl ether sulfate salt, acyl sarcosinate, and sulfosuccinate; cationic surfactants such as alkyl trimethyl ammonium salts, dialkyl dimethyl ammonium salts, alkyl pyridinium salts, and benzalkonium chloride; and ampholytic surfactants such as imidazoline surfactants and betaine surfactants.

Specific examples of the oily components include, but are not limited to: plant oils such as olive oil, jojoba oil, castor oil, rice bran oil, and palm oil; animal oils such as squalane, beef tallow, and lanoline; synthetic oils such as silicone oil, polyisobutene, fatty acid ester, and fatty acid glycerin; waxes such as beeswax, Japan wax, candelilla wax, and carnauba wax; hydrocarbons such as liquid paraffin, ceresin, micro-crystalline wax, and vaseline; higher alcohols such as cetanol, stearyl alcohol, and octyl dodecanol; higher fatty acids such as stearic acid, lauric acid, myristic acid, and oleic acid; and silicone resins, silicone rubbers, polyether-modified silicon, and perfluoroether.

Specific examples of the colorants include, but are not limited to, organic dyes and natural dyes such as Brilliant Blue FCF, Fast Green FCF, Lithol Rubine BCA, Fast Acid Magenta, Tartrazine, chlorophyll, and β-carotene. Each of these colorants can be used alone or in combination with others.

Specific examples of the moisturizing agents include, but are not limited to, vitamin A, B, C, and E and derivatives thereof, amino acids, sodium hyaluronate, and trimethylglycine.

### [3] Adhesive Layer

The members constituting the transdermal device may be adhered to each other via an adhesive layer.

Referring to FIGS. 1A, 1B, 2, and 3, the sheet-shaped electrode 21 is adhered to and laminated on the carrier layer 11 via the adhesive layer 12.

Other functional layers (including sheets and films), to be described later, may also be adhered via an adhesive layer.

The adhesive layer 12 may be formed by applying a solution of an adhesive (including a pressure-sensitive adhesive) by means of coating, followed by drying. The adhesive layer 12 may also be made of a hot-melt film.

Preferably, the adhesive comprises a material that exerts a sufficient adhesive force even when being formed into a thin layer.

Specific examples of the pressure-sensitive adhesive include, but are not limited to, natural rubbers, synthetic rubbers and elastomers, vinyl chloride-vinyl acetate copolymers, polyvinyl alkyl ethers, polyacrylates, modified-polyolefin-resin-based pressure-sensitive adhesives, and curable pressure-sensitive adhesives to which a curing agent (e.g., isocyanate) is added. Among these, curable pressure-sensitive adhesives are preferable for adhering polyolefin foams or polyester films.

Specific examples of the adhesive include, but are not limited to, dry lamination adhesives which mixes a polyurethane resin solution and a polyisocyanate resin solution, styrene-butadiene-rubber-based adhesives, and epoxy-based two-component (e.g., epoxy resin and polythiol, epoxy resin and polyamide) curable adhesives. In particular, solution-type adhesives and epoxy-based two-component curable adhesive are preferable, and those being transparent are more preferable. Adhesives which are capable of improving adhesive force by using an appropriate adhesive primer are preferably used in combination with the adhesive primer.

The adhesive layer 12 may be disposed only partially (for example, only at the periphery), as illustrated in FIGS. 2 and 3.

Alternatively, the electrode 21 may be directly stacked on the carrier layer 11 without using any adhesive. In this case, the electrode 21 can be tightly adhered to the carrier layer 11 by being fixed with a medical tape or bandage.

In the present embodiment, the adhesive layer 12 is disposed on a plane on which the sheet-shaped electrode 21 is formed, and the carrier layer 11 is further stacked on the adhesive layer 12. Alternatively, the adhesive layer 12 may be disposed on another plane on which no electrode is formed, and the carrier layer 11 is further stacked on the adhesive layer 12.

In the latter case, when a low frequency (as applied in a low-frequency therapy equipment) is employed, a desired active ingredient can be supplied to the skin based on the principle of accumulating electricity in the substrate of the electrode by application of a pulse current and thereby passing a current by the action of electric charge induced in the living body (skin).

### [4] Functional Layer

The transdermal device may further include other functional layers such as a stress relaxation layer and a sticking layer.

Examples of the functional layers include, but are not limited to, optical adjustment layer, anti-Newton ring layer, anti-glare layer, matting agent layer, protective layer, charge prevention layer, smoothing layer, adhesion improving layer, light shielding layer, anti-fogging layer, anti-fouling layer, and print layer, which have been conventionally used.

### Stress Relaxation Layer

The stress relaxation layer prevents quality deterioration. Specifically, the stress relaxation layer prevents the members (e.g., films) having different thermal expansion coefficients and being attached to each other from being detached from each other even during an environmental loading test.

### Sticking Layer

As illustrated in FIGS. 1A, 1B, and 2, a sticking layer 13 may be disposed covering the edge part of the carrier layer 11. FIG. 1B is a cross-sectional view of the transdermal device 20 illustrated in FIG. 1A.

Preferably, the sticking layer 13 is disposed for the purpose of bringing the transdermal patch 10 into intimate contact with the skin, especially when the transdermal patch 10 has a large area. In a case in which the transdermal patch 10 by itself is able to remain intimate contact with the skin, the sticking layer 13 can be omitted.

The sticking layer 13 may comprise a biocompatible adhesive material.

Specific examples of such a material include, but are not limited to: gels containing natural polymers, such as agar, gelatin, agarose, xanthane gum, gellan gum, sclerotium gum, gum arabic, gum tragacanth, gum karaya, cellulose gum, tamarind gum, guar gum, locust bean gum, glucomannan, chitosan, carrageenan, quince seed, galactan, mannan, starch, dextrin, curdlan, casein, pectin, collagen, fibrin, peptide, chondroitin sulfates (e.g., sodium chondroitin sulfate), hyaluronic acid (mucopolysaccharide), hyaluronates (e.g., sodium hyaluronate), alginic acid, alginates (e.g., sodium alginate, calcium alginate), and derivatives thereof: gels containing cellulose derivatives (e.g., methyl cellulose, hydroxymethyl cellulose, hydroxyethyl cellulose, hydroxypropyl cellulose, hydroxypropyl methyl cellulose, carboxymethyl cellulose) and salts thereof; gels containing polyacrylic acid or polymethacrylic acid (e.g., polyacrylic acid, polymethacrylic acid, alkyl copolymers of acrylic acid and methacrylic acid) and salts thereof; gels containing synthetic polymers such as polyvinyl alcohol, polyhydroxyethyl methacrylate, polyacrylamide, poly(N-isopropylacrylamide), polyvinyl pyrrolidone, polystyrene sulfonic acid, polyethylene glycol, carboxyvinyl polymer, alkyl-modified carboxyvinyl polymer, maleic anhydride copolymer, polyalkylene-oxide-based resin, N-vinylacetamide cross-linked body, acrylamide cross-linked body, and starch-acrylate graft copolymer cross-linked product; silicone hydrogel; interpenetrating-network-structure or semi-interpenetrating-network-structure hydrogel; and combinations thereof. In particular, for improving load resistance and biocompatibility, gels containing collagen and/or glucomannan, gels containing carboxymethyl cellulose and/or carboxymethyl cellulose sodium, gels containing polyacrylic acid and/or sodium polyacrylate, and interpenetrating-network-structure or semi-interpenetrating-network-structure hydrogel are preferable.

As the hydrogel, those comprising a polymer are generally used, but the hydrogel is not limited thereto.

### [5] Electrode

The electrode 21 is a sheet-shaped electrode comprising a sheet material and an electrode material.

Examples of the electrode material include a silver/silver chloride electrode that is generally used for iontophoresis. When there is a concern about metallic allergy, a conductive material (e.g., hydrocarbon material) other than metal is preferably used.

Examples of the electrode material further include a conductive paste obtainable by kneading a binding agent (organic binding agent), in which a conductive material is dissolved in an organic solvent, into a paste.

Specific examples of the conductive material include, but are not limited to: carbon materials such as carbon nanotube, carbon black, Ketjen black, glassy carbon, graphene, fullerene, carbon fiber, carbon fabric, and carbon aerogel; conductive polymers such as polyaniline, polyacetylene, polypyrrole, poly(p-phenylenevinylene), polythiophene, and poly(p-phenylene sulfide); semiconductors such as silicone, germanium, indium tin oxide (ITO), titanium oxide, copper oxide, and silver oxide; and metals such as gold, platinum, titanium, aluminum, tungsten, copper, iron, and palladium. Among these, carbon fabric and carbon nanotube are preferable in terms of flexibility and electrochemical stability.

Examples of the sheet material include a flexible film, preferably having stretchability. More specifically, resin films generally used for printed electronics can be used.

Specific examples of such films include, but are not limited to, urethane film, polyethylene terephthalate film, polyethylene naphthalate film, polycarbonate film, polyimide film, syndiotactic polystyrene film, and polyphenylene sulfide film. In particular, a urethane film or silicone rubber sheet is preferably used when the sheet material is desired to have stretchability. In a case in which the sheet material is a glass substrate, it is preferable that several glass substrates already having a wiring are arranged in a distributed manner so as not to degrade skin followability, rather than forming a glass substrate covering the whole surface of the carrier layer.

The electrode 21 may be formed by creating a circuit on one surface of the sheet material. The circuit can be created by a known method such as printing and vapor deposition. Alternatively, the circuit may be a wiring of a printed electronics. In a case in which the circuit is created by inkjet, a primer coating layer is formed on the sheet material prior to formation of a wiring.

When the electrode 21 is formed by designing a wiring corresponding to a desired application site utilizing printed electronics technology, the active ingredient can be selectively administrated thereto.

### [6] Power Source

Examples of the power source 22 include known power supplies, such as a dye sensitized solar cell, current generator, and wireless power feeder.

### [7] Injection of External Composition

How to introduce the external composition 40 into the carrier layer 11 is determined depending on the properties (e.g., viscosity) of the external composition 40. For example, when the viscosity is high, the external composition 40 may be introduced by vacuum injection, and when the viscosity is low, the external composition 40 may be dropped into the carrier layer 11.

Before introducing the external composition 40 into the carrier layer 11, it is preferable that the members (e.g., the hollow structure 30) constituting the carrier layer 11 are sterilized. The sterilization treatment can be performed by a known method appropriately selected according to the properties of the material of the hollow structure 30.

In a case in which the hollow structure 30 is formed of an ultraviolet curable resin, ultraviolet irradiation sterilization or ultraviolet ozone cleaning is preferably performed for eliminating residual unreacted monomers.

### EXAMPLES

### Example 1

A transdermal patch and a transdermal device were prepared in the following manner.

The prepared transdermal patch was subjected to evaluations of skin followability and liquid retainability. The prepared transdermal device was subjected to an evaluation of external composition permeability to the application target.

### Preparation of Transdermal Patch

A hollow structure (honeycomb structure) was prepared with a ultraviolet curable resin according to a method described in JP-4678731-B (corresponding to JP-2007-0989830-A) as follows.

First, an ultraviolet curable resin (“material”) was applied to a protective material to prepare a base material. The base material was put on a template having independent recesses and attached thereto from the edge part thereof using an attachment jig so that no bubbles enter. During the attachment process, a pressure control was performed so that the material would not excessively adhere to the recesses.

A container containing the base material and the template was decompressed by a decompressor for 90 seconds so that gas contained inside the recesses relatively generated a pressure, thereby forming hollow portions (cells) in the material. Since the material in intimate contact with the template did not move, the hollow portions were kept independent. After the partition wall of the cells had grown to have a desired height, ultraviolet rays were emitted thereto to cure the material within which the cells had been formed.

After the base material having a honeycomb shape was detached from the template, the closed surface thereof was cut to obtain a honeycomb structure having cells penetrating through one plane surface to the other plane surface.

The resulting honeycomb structure had a volume of 2 mm³.

The thickness of the partition wall of the cells was 1 μm, the pitch of the cells was 50 μm, and the height of the cells was 500 μm, as shown in Table 1.

### Preparation of Transdermal Device

On a polyurethane film, a silver/silver chloride paste (5880 available from Du Pont) was printed through a 290 mesh screen having a pattern thereon. The printed film was dried at 120 degrees C. for 10 minutes. Thus, a sheet-shaped electrode (hereinafter “electrode sheet”) was prepared.

An extraction electrode was provided to the electrode sheet for connecting the electrode sheet to a power source.

An adhesive layer having a width of 0.5 cm was formed along the outer periphery of the electrode sheet. The adhesive layer was prepared by cutting a hot melt film (HM30 available from PANAC Co., Ltd.) into pieces.

After the hot melt film was laminated on the surface of the electrode sheet on which a circuit had been formed, they were heated to 60 degrees C. by a hot plate and the hot melt film was pressurized by a roller to be brought into intimate contact with the electrode sheet. The honeycomb structure was stacked on the surface on which the hot melt film was disposed, and they were heated to 60 degrees C. again by a hot plate. The honeycomb structure was pressurized by a roller to be brought into intimate contact with the electrode sheet.

A voltage/current generator (DC Voltage/Current Source/Monitor 6242 available from ADC CORPORATION), serving as the power source, was connected to the extraction electrode of the electrode sheet to pass an electrical current.

As a model dispersion liquid for evaluating external composition permeability, an aqueous dispersion liquid of a liposome containing calcein was prepared. The liposome was prepared as follows.

First, desired concentration and volume of a lipid were determined, and the required amount of a preservation solution of the lipid was calculated. A vial container having an appropriate size was prepared. The required amount of a chloroform solution of the lipid was sampled with a pipette (microsyringe) and contained in the vial container. After the chloroform was evaporated from the solution with a nitrogen gas sprayer, the vial container was left at rest in a vacuum desiccator for at least one hour for completely evaporating residual chloroform. Distilled water was thereafter poured in the vial container and calcein was added thereto. The vial container was held in an ultrasonic water tank so that the liquid level in the vial container became lower than that in the ultrasonic water tank, and exposed to ultrasonic waves at the maximum output or about 30 seconds. The vial container was taken out of the water tank and immediately stirred with a test tube mixer vigorously. The above operation was repeated 3 to 4 times so that the lipid films were detached from the wall surface. Thus, a liposome containing calcein was obtained.

The resulting aqueous dispersion liquid of the liposome was dropped on the honeycomb structure with a syringe so as to be carried in the cells.

### Evaluation Test 1

Skin followability of the transdermal patch was evaluated in the following manner.

The above-prepared transdermal patch comprising the honeycomb structure as the carrier layer was cut into a piece having an area of 70 mm×100 mm. This piece was applied to an elbow of a subject. The subject had been selected from those who have used conventional transdermal patches or transdermal devices.

### (1) Sensory Test

As an indicator for skin followability, the ease of moving the site to which the patch was applied was evaluated (based mainly on the feelings of bondage and tightness) according to the following criteria. The evaluation result shown in Table 1 was the average among ten subjects.

Evaluation Criteria

5: Easy to move

4: Slightly easy to move

3: Neither easy nor hard to move

2: Slightly hard to move

1: Hard to move

### (2) Adhesiveness

Three hours after the transdermal patch was applied to the elbow, the transdermal patch was visually observed to determine the degree of floating or peeling. As an indicator for skin followability, adhesiveness to the skin (difficulty in peeling from skin) was evaluated according to the following criteria.

The evaluation result shown in Table 1 was the average among five subjects.

Evaluation Criteria

5: The patch was adhered to the elbow. Neither floating nor peeling was observed.

4: Floating or peeling was observed at the edge part of the patch. The floating or peeling area was less than 5%.

3: Floating or peeling was observed at the edge part of the patch. The floating or peeling area was not less than 5% and less than 10%.

2: Floating or peeling was observed at the edge part of the patch. The floating or peeling area was not less than 10% and less than 20%.

1: Floating or peeling was observed at the edge part of the patch. The floating or peeling area was 20% or more.

### Evaluation Test 2

Liquid retainability of the transdermal patch (honeycomb structure) was evaluated in the following manner. Here, the liquid retainability indicates the degree of transpiration (drying) of the carried dispersion liquid.

The evaluation was performed using the above-prepared model dispersion liquid, i.e., the aqueous dispersion liquid of the liposome containing the calcein.

The transdermal patch comprising the carrier layer (honeycomb structure) having an area of 2 cm×2 cm sufficiently carrying the model dispersion liquid was stored in a heating cabinet having a temperature of 40° C. The weight of the transdermal patch was measured before and after the storage. The residual rate (%) of the carried model dispersion liquid was determined from the following formula.

Residual Rate (%)=(Weight After Storage)/(Weight Before Storage)×100

The results are shown in Table 1.

The larger the residual rate, the greater the liquid retainability. From the viewpoint of practical utility, preferably, the residual rate is 40% or more. When liquid retainability is high, the loss of electricity is suppressed and electrophoretic property and permeability can be improved.

### Evaluation Test 3

Model dispersion liquid permeability to the application target of each transdermal device was evaluated in the following manner.

As a model of the application target, a gel sheet was prepared. The gel sheet was a gel having a final concentration of 3% obtained by diluting an agarose gel (Agarose KANTO available from Kanto Chemical Co., Inc.) with 1×TAE buffer.

The transdermal device having an area of 1 cm×1 cm sufficiently carrying the model dispersion liquid was adhered to the gel sheet and disposed on a positive electrode side. A current of 0.45 mA/cm² was passed under an environment of 20° C. The distance between the positive electrode and the negative electrode was 3 cm. The positive electrode side of the agarose gel (the gel sheet) was cut out and visually observed to measure the permeation distance at immediately below the electrode. The permeation distance refers to the moving distance of the nanoparticle (i.e., liposome containing calcein) observed visually or with a fluorescent microscope. Permeability was evaluated based on the moving distance according to the following criteria. The results were shown in Table 1.

Evaluation Criteria

A: The moving distance of liposome after a lapse of 20 minutes was 1.5 cm or more.

B: The moving distance of liposome after a lapse of 20 minutes was 1 cm or more and less than 1.5 cm.

C: The moving distance of liposome after a lapse of 20 minutes was 0.5 cm or more and less than 1 cm.

D: The moving distance of liposome after a lapse of 20 minutes was less than 0.5 cm.

### Example 2

The procedure for preparing and evaluating a transdermal patch and a transdermal in Example 1 was repeated except for changing the thickness of the partition wall and the height of the cells in the honeycomb structure to 22 μm and 50 μm, respectively.

The results were shown in Table 1.

### Example 3

The procedure for preparing and evaluating a transdermal patch and a transdermal device in Example 1 was repeated except for replacing the model dispersion liquid with another one being an aqueous dispersion liquid of a micelle containing a lipid-soluble fluorescent dye. The results were shown in Table 1.

The micelle was prepared as follows.

The micelle was prepared with a biodegradable PEG polyester diblock copolymer (mPEG-PLGA(5,000 Da-10,000 Da), product No. 765139 of Sigma-Aldrich).

Specifically, mPEG-PLGA was dissolved in dichloromethane so that the concentration became 5% (w/v).

Rhodamine B was heated in n-butanol solvent in the presence of p-toluenesulfonic acid. In the succeeding extraction treatment, sodium p-toluenesuofonate was added to synthesize a lipid-soluble fluorescent dye. The lipid-soluble fluorescent dye was dissolved in dichloromethane so that the concentration became 2.5% (w/v).

Next, 1 mL of the mPEG-PLGA solution and 0.1 mL of the fluorescent dye solution were mixed to prepare a mixture solution.

A scintillation vial was filled with 20 mL of distilled water and the mixture solution was dropped therein while the distilled water was stirred by an overhead stirrer at a revolution of 2,000 rpm. The stirring was continued for one hour or more. The resulting mixture was filtered with a 0.45-μm PVDF filter, thus obtaining a nanoparticle (micelle).

### Example 4

The procedure for preparing and evaluating a transdermal patch and a transdermal device in Example 1 was repeated except for replacing the model dispersion liquid with another one being a dispersion liquid of a liposome containing calcein, the dispersion medium of which being an electrolyte aqueous solution. The results were shown in Table 1.

The electrolyte aqueous solution was prepared as follows.

First, 8 g of sodium chloride (having a final concentration of 137 mmol/L), 0.2 g of potassium chloride (having a final concentration of 2.68 mmol/L), 1.44 g of di sodium hydrogen phosphate (having a final concentration of 10 mmol/L), and 0.24 g of potassium dihydrogen phosphate (having a final concentration of 2 mmol/L) were dissolved in 900 mL of ultrapure water, and the pH was adjusted to 7.4 with hydrochloric acid. The mixture was diluted to 1 L in measuring cylinder and sterilized in an autoclave, thus obtaining an electrolyte aqueous solution.

### Comparative Example 1

The procedure for preparing and evaluating a transdermal patch and a transdermal device in Example 1 was repeated except for replacing the hollow structure with a medical unwoven fabric (SPAN CLOTH available from APOLLO EIZAI Co., Ltd.). The results are shown in Table 1.

### Comparative Example 2

The procedure for preparing and evaluating a transdermal patch and a transdermal device in Example 1 was repeated except for replacing the model dispersion liquid with a 10-mM mixture liquid of water and calcein without being contained in liposome. The results are shown in Table 1.

TABLE 1

| | | | | | | Comparative | Comparative |
|---|---|---|---|---|---|---|---|
| | | Example 1 | Example 2 | Example 3 | Example 4 | Example 1 | Example 2 |
| Hollow | Thickness of | 1 | 22 | 1 | 1 | — | 1 |
| Structure | Partition | | | | | | |
| | Wall of | | | | | | |
| | Cells (μm) | | | | | | |
| | Pitch of | 50 | 50 | 50 | 50 | — | 50 |
| | Cells (μm) | | | | | | |
| | Height of | 500 | 50 | 500 | 500 | — | 500 |
| | Cells (μm) | | | | | | |
| Nanoparticle | Type of | Liposome | Liposome | Micelle | Liposome | — | — |
| | Nanoparticle | | | | | | |
| | Particle | 100 | 100 | 100 | 100 | — | — |
| | Diameter | | | | | | |
| | (nm) | | | | | | |
| Dispersion | Type of | Water | Water | Water | Electrolyte | — | — |
| Medium | Dispersion | | | | Aqueous | | |
| | Medium | | | | Solution | | |
| Evaluations | Evaluation | 4 | 1 | 4 | 4 | 1 | 4 |
| | Test 1(1) | | | | | | |
| | Evaluation | 4 | 1 | 4 | 4 | 1 | 4 |
| | Test 1(2) | | | | | | |
| | Evaluation | 46% | 40% | 42% | 51% | 11% | 38% |
| | Test 2 | | | | | | |
| | Evaluation | B | B | B | A | D | D |
| | Test 3 | | | | | | |

As indicated in Table 1, the transdermal device and transdermal patch in accordance with some embodiments of the present invention provide excellent skin followability and liquid retainability. Thus, the loss of electricity can be reduced. In particular, due to high skin followability, the air is prevented from entering from the applied surface and therefore the occurrence of insulation is also prevented. Furthermore, uncomfortable feeling of the wearer can be reduced, which is preferable for a long-term application to the wearer.

In addition, as indicated in Table 1, the transdermal device in accordance with some embodiments of the present invention provides excellent permeability. When the carrier layer comprises the hollow structure prepared above and the external composition comprises the above dispersion liquid of the nanoparticle, the nanoparticle is given excellent electrophoretic property and the active ingredient can sufficiently permeate the application site because the dispersion liquid is sufficiently carried in the carrier layer.

Numerous additional modifications and variations are possible in light of the above teachings. It is therefore to be understood that, within the scope of the above teachings, the present disclosure may be practiced otherwise than as specifically described herein. With some embodiments having thus been described, it will be obvious that the same may be varied in many ways. Such variations are not to be regarded as a departure from the scope of the present disclosure and appended claims, and all such modifications are intended to be included within the scope of the present disclosure and appended claims.

1. A transdermal device comprising:
a transdermal patch comprising a carrier layer, the carrier layer comprising:
a hollow structure comprising a plurality of cells partitioned by an insulator, the hollow structure having hollow portions penetrating through one plane surface to the other plane surface of the hollow structure; and
an external composition comprising a dispersion liquid comprising a nanoparticle containing an active ingredient;
a sheet-shaped electrode stacked overlying one surface of the transdermal patch; and
a power source connected to the sheet-shaped electrode.

2. The transdermal device of claim 1, wherein each one of the plurality of cells forms a flow path having a substantially linear shape.

3. The transdermal device of claim 1, wherein the insulator forms a partition wall partitioning the cells adjacent to each other, the partition wall having a thickness of from 0.1 to 30 μm.

4. The transdermal device of claim 1, wherein the hollow structure is a honeycomb structure.

5. The transdermal device of claim 1, wherein the hollow structure further comprises a plurality of hollow needles on a surface contactable with skin, the hollow needles with the respective cells.

6. The transdermal device of claim 1, wherein the dispersion liquid further comprises a dispersion medium comprising an electrolyte aqueous solution.

7. The transdermal device of claim 1, wherein the nanoparticle comprises at least one of liposome and micelle.

8. A transdermal patch comprising:
a carrier layer comprising:
a hollow structure comprising a plurality of cells partitioned by an insulator, the hollow structure having hollow portions penetrating through one plane surface to the other plane surface of the hollow structure; and
an external composition comprising a dispersion liquid comprising a nanoparticle containing an active ingredient.
